(19) United States
(12) Patent Application Publication
(10) Pub. No.: US 2010/0028914 A1
(43) Pub. Date: Feb. 4, 2010

(54) ASSESSING LUNG NODULES

(75) Inventors: Otis B. Rickman, Rochester, MN (US); Nicholas E. Vlahakis, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/293,318

(22) PCT Filed: Mar. 16, 2007
(86) PCT No.: PCT/US2007/064231

§ 371 Date: Sep. 4, 2009

Related U.S. Application Data
(60) Provisional application No. 60/783,345, filed on Mar. 17, 2006.

(51) Int. Cl. G01N 33/574

(52) U.S. Cl. 435/7.23

(57) ABSTRACT

This document provides methods and materials related to distinguishing malignant solitary pulmonary nodules from benign solitary pulmonary nodules in mammals (e.g., humans).

### BACKGROUND

1. Technical Field

This document relates to methods and materials involved in determining whether solitary pulmonary nodules in mammals (e.g., humans) are benign or malignant.

2. Background Information

Lung cancer is the leading cause of cancer deaths in both men and women, accounting for about 29 percent of all cancer deaths. Lung cancer is one of the cancers that show the greatest difference in survival between early and late diagnosis. Based in part on late stage diagnosis, the current five-year survival rate for lung cancer is 15 percent. For cancers detected in the early stage (Stage IA), however, five-year survival is greater than 80 percent. Chest radiography or CT scanning can detect solitary pulmonary nodules (SPNs). However, SPNs represent a clinical dilemma for the patient and clinician. Although most SPNs are benign, primary malignancy can be found in about 35 percent of SPNs, and solitary metastases can account for another 23 percent. The probability of malignancy varies considerably despite use of Bayes' theorem, logistic regression models, and neural network analysis.

### SUMMARY

This document provides methods and materials related to distinguishing a malignant SPN from a benign SPN in a mammal (e.g., a human). As described herein, the expression of αᵥβ₃ integrin polypeptides on malignant SPNs and the expression of little, or no, αᵥβ₃ integrin polypeptides on benign SPNs can be used to distinguish malignant SPNs from benign SPNs. In some cases, a malignant SPN can be distinguished from a benign SPN using a labeled molecule that binds to cells expressing αᵥβ₃ integrin polypeptides. Having the ability to distinguish between malignant and benign SPNs can help clinicians to manage therapeutic intervention effectively and relieve patients from the psychological burden of not knowing whether their nodule or nodules are benign or malignant.

In general, one aspect of this document features a method for assessing a solitary pulmonary nodule. The method comprises, or consists essentially of, determining whether or not the solitary pulmonary nodule expresses an αᵥβ₃ integrin polypeptide, wherein the expression of the αᵥβ₃ integrin polypeptide indicates that the solitary pulmonary nodule is malignant, and wherein the expression of little or no αᵥβ₃ integrin polypeptide indicates that the solitary pulmonary nodule is benign. The solitary pulmonary nodule can be a human solitary pulmonary nodule. The determining step can comprise, or consist essentially of, contacting tissue of the solitary pulmonary nodule with a labeled molecule having the ability to bind to the αᵥβ₃ integrin polypeptide. The labeled molecule can be an antibody. The labeled molecule can be an RGD peptide. The labeled molecule can be ^{99m}Tc-NC100692. The labeled molecule can be administered to a mammal having the solitary pulmonary nodule.

In another embodiment, this document features a method for assessing a solitary pulmonary nodule. The method comprises, or consists essentially or, determining whether or not the solitary pulmonary nodule lacks expression of an αᵥβ₃ integrin polypeptide, wherein the lack of expression of the αᵥβ₃ integrin polypeptide indicates that the solitary pulmonary nodule is benign. The solitary pulmonary nodule can be a human solitary pulmonary nodule. The determining step can comprise, or consist essentially of, contacting tissue of the solitary pulmonary nodule with a labeled molecule having the ability to bind to the αᵥβ₃ integrin polypeptide. The labeled molecule can be an antibody. The labeled molecule can be an RGD peptide. The labeled molecule can be ^{99m}Tc-NC100692. The labeled molecule can be administered to a mammal having the solitary pulmonary nodule.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides methods and materials related to determining whether or not an SPN in a mammal is malignant. Typically, an SPN is a lung lesion less than three centimeters in diameter that is surrounded by normal lung tissue and is not associated with adenopathy or atelectasis. SPNs can be detected in mammals by any method, such as chest radiography or CT scanning. An SPN in a mammal can be malignant or benign. A malignant SPN can be any type of primary lung cancer, such as squamous cell carcinoma, small cell carcinoma, adenocarcinoma, or large cell carcinoma, or metastatic cancer. A benign SPN can be any type of benign lung nodule including, without limitation, a granuloma or a hamartoma.

A malignant SPN can be distinguished from a benign SPN in any mammal having an SPN. For example, malignant SPNs can be distinguished from benign SPNs in a human, dog, monkey, mouse, or rat. In some cases, malignant SPNs can be distinguished from benign SPNs in a smoker or a mammal with a genetic predisposition to develop lung cancer. In some cases, it can be determined whether or not a mammal suspected of having pulmonary nodule, such as a coal miner, has a malignant SPN, a benign SPN, or both.

The expression of αᵥβ₃ integrin polypeptides on malignant SPNs and the expression of little, or no, αᵥβ₃ integrin polypeptides on benign SPNs can be used to distinguish between malignant SPNs and benign SPNs. Any method can be used to determine whether or not αᵥβ₃ integrin polypeptides are expressed on SPNs. For example, SPN tissue can be contacted with a molecule that binds to cells expressing αᵥβ₃ integrin polypeptides. The level of binding of such a molecule to the SPN tissue can be detected. The presence of the molecule bound to the tissue can indicate that the tissue has αᵥβ₃ integrin polypeptides and is malignant. The absence of the molecule bound to the tissue or the presence of a barely detectable level of the molecule bound to the tissue can indicate that the tissue expresses no, or low level of, αᵥβ₃ integrin polypeptides and is benign.

An αᵥβ₃ integrin polypeptide can be any integrin polypeptide having αᵥ and β₃ polypeptide subunits. Examples of integrin αᵥ polypeptides include human integrin αᵥ polypeptides (e.g., human integrin as polypeptides set forth under GenBank® GI numbers gi|4504763 or gi|466372), mouse integrin αᵥ polypeptides (e.g., mouse integrin αᵥ polypeptides set forth under GenBank® GI numbers gi|6680486, gi|122889688, gi|22889687, gi|122889686, gi|122889685, or gi|4432991), monkey integrin αᵥ polypeptides (e.g., monkey integrin αᵥ polypeptide set forth under GenBank® GI number gi|109100308), chimpanzee integrin αᵥ polypeptides (e.g., chimpanzee integrin αᵥ polypeptide set forth under GenBank® GI number gi|114582305), and cattle integrin αᵥ polypeptides (e.g., cattle integrin αᵥ polypeptides set forth under GenBank® GI numbers gi|12831630 or gi|27805957). Examples of integrin β₃ polypeptides include human integrin β₃ polypeptides (e.g., human integrin β₃ polypeptides set forth under GenBank® GI numbers gi|7690082, gi|54124349, or gi|386833), chicken integrin β₃ polypeptides (e.g., chicken integrin β₃ polypeptides set forth under GenBank® GI numbers gi|452854 or gi|46048954), and dog integrin β₃ polypeptides (e.g., dog integrin β₃ polypeptide set forth under Genbank® GI number gi|50978890). Examples of integrin αᵥ and β₃ polypeptides also include variants of integrin αᵥ and β₃ polypeptides (e.g., polypeptides having mutational differences such as single nucleotide polymorphisms) as well as homologs and orthologs of integrin αᵥ and β₃ polypeptides.

Any molecule that binds to cells having αᵥβ₃ integrin polypeptides and that can be detected when bound to αᵥβ₃ integrin polypeptides on cells can be used to assess the presence, absence, or low level of αᵥβ₃ integrin polypeptides on SPNs. For example, RGD peptides, small molecule αᵥβ₃ integrin antagonists, lectins, or anti-αᵥβ₃ integrin antibodies can be used to determine whether or not αᵥβ₃ integrin polypeptides are expressed by SPNs.

An RGD peptide can be any polypeptide comprising an arginine, glycine, aspartic acid amino acid sequence. Examples of RGD peptides include, without limitation, ^{99m}Tc-NC100692, SU015 (Mousa, J Cardiovasc. Pharmacol., 45:462 (2005), and ¹²⁵I-c(RGD(l)yV) (Lee el at., J Nucl. Med., 46:472 (2005)). An RGD peptide can be a monomeric polypeptide or a multimeric (e.g., dimeric) polypeptide. An RGD peptide also can be cyclic and can be stabilized (e.g., by disulfide bonds). In some cases, an RGD peptide can contain one or more modifications. For example, an RGD peptide can be modified to be pegylated or covalently attached to oligomers, such as short, amphiphilic oligomers that enable oral administration or improve the pharmacokinetic or pharmacodynamic profile of a conjugated RGD peptide. The oligomers can comprise water soluble polyethylene glycol (PEG) and lipid soluble alkyls (short chain fatty acid polymers). See, for example, International Patent Application Publication No. WO 2004/047871. In some cases, an RGD peptide can be incorporated in liposomes.

Examples of small molecule αᵥβ₃ integrin antagonists include: (S)-3-Oxo-8-[2-[6-(methylamino)-pyridin-2-yl]-1-ethoxy]-2-(2,2,2-trifluoroethyl)-2,3,4,5-tetrahydro-1H-2-benzazepine-4-acetic acid and 3-[2-oxo-3-[3-(5,6,7,8-tetrahydro-[1,8]naphthyridin-2-yl) propyl]-imidazolidin-1-yl]-3(S)-(6-methoxy-pyridin-3-yl) propionic acid.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgGl, IgG4, or IgA2), or subclass. In addition, an antibody can be from any animal including birds and mammals. For example, an antibody can be human, rabbit, sheep, or goat. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-αᵥβ₃ integrin antibody can bind to cells having αᵥβ₃ integrin polypeptides at an affinity of at least 10⁴ mol⁻¹, e.g., at least 10⁵, 10⁶, 10⁷, 10⁸, 10⁹, 10¹⁰, 10¹¹, or 10¹² mol⁻¹. Examples of commercially available anti-αᵥβ₃ integrin antibodies include Integrin alpha V beta 3 antibody [23C6] (code ab20143; Abcam, Cambridge, Mass.) and Integrin alpha V beta 3 antibody [BV3] (code ab7166; Abcam).

A molecule that binds to cells having αᵥβ₃ integrin polypeptides can be labeled for detection. For example, such a molecule can be labeled with a radioactive molecule, a metal radionuclide, a paramagnetic metal ion, a fluorescent molecule, a fluorescent metal ion, a heavy metal ion, a cluster ion, technetium-99m, or ⁶⁴Cu. A molecule capable of binding to cells having αᵥβ₃ integrin polypeptides can also be detected indirectly using a labeled molecule that binds to the molecule that binds to cells having αᵥβ₃ integrin polypeptides. For example, an anti-αᵥβ₃ integrin antibody that binds to cells having αᵥβ₃ integrin polypeptides can be detected using a labeled secondary antibody.

Any method can be used to determine whether or not a molecule binds to cells expressing αᵥβ₃ integrin polypeptides. For example, a binding assay, such as a radioligand binding assay, can be used to determine whether or not a molecule binds to cells having αᵥβ₃ integrin polypeptides, such as angiogenic endothelial cells. A binding assay can also be used to determine whether or not a molecule binds to cells that express little or no αᵥβ₃ integrin polypeptides including, but not limited to, normal lymphatic vessel cells or normal brain cells. The affinity at which a molecule binds to cells having or not having αᵥβ₃ integrin polypeptides can be determined in competitive binding experiments using a molecule known to have a high affinity for αᵥβ₃ integrin polypeptides, such as echistatin. Binding affinities can also be measured using surface plasmon resonance. A molecule that binds to cells having αᵥβ₃ integrin polypeptides can have a Kᵢ of less than 1000 nm, e.g., less than 900 nm, 800 nm, 700 nm, 600 nm, 500 nm, 400 nm, 300 nm, 200 nm, 100 nm, or 50 nM, in a competitive binding assay for αᵥβ₃ integrin polypeptides.

A molecule that binds to cells having αᵥβ₃ integrin polypeptides can lack the ability to bind to cells not having αᵥβ₃ integrin polypeptides. In some cases, a molecule that binds to cells having αᵥβ₃ integrin polypeptides can have the ability to bind to cells not expressing αᵥβ₃ integrin polypeptides, but at an affinity at least 1-fold less (e.g., at least 1-fold, 1.5-fold, 2-fold, 3-fold, 4-fold, 5-fold, 6-fold, 7-fold, 8-fold, 9-fold, or 10-fold less) than the affinity at which the molecule binds to cells having αᵥβ₃ integrin polypeptides under the same conditions.

A molecule, e.g., a labeled molecule, that binds to cells having αᵥβ₃ integrin polypeptides can be used to determine whether an SPN in a mammal is malignant or benign. For example, such a labeled molecule can be administered to a mammal, and an image of the mammal's lungs can be generated to determine whether or not the molecule is bound to an SPN. The labeled molecule can be administered systemically or directly to the lungs. The administration can be performed in any manner, such as by intravenous injection or inhalation. An image of the lungs can be generated using any technology, including, without limitation, positron emission tomography (PET). The level of binding of the labeled molecule to SPN tissue can be used to determine whether the SPN tissue is malignant or benign. For example, the presence of the labeled molecule bound to SPN tissue can indicate that the SPN tissue is malignant, and the presence of little or no labeled molecule bound to SPN tissue can indicate that the SPN tissue is benign. In some cases, a detectable level of binding of the labeled molecule bound to SPN tissue can indicate that the SPN tissue is malignant, and an undetectable level of binding of the labeled molecule bound to SPN tissue can indicate that the SPN tissue is benign.

In some cases, the presence of an elevated level of expression of αᵥβ₃ integrin polypeptides by SPN tissue can indicate that the SPN tissue is malignant, and the absence of an elevated level of expression of αᵥβ₃ integrin polypeptides by SPN tissue can indicate that the SPN tissue is benign. The term “elevated level” as used herein with respect to the level of expression of αᵥβ₃ integrin polypeptides by SPN tissue is any level of expression that is greater than a reference level, which is the level of expression of αᵥβ₃ integrin polypeptides typically detected in benign SPN tissue, For example, a reference level of expression of αᵥβ₃ integrin polypeptides can be the average level of expression of αᵥβ₃ integrin polypeptides by benign SPNs in a random sampling of mammals (e.g., 5, 10, 25, or 50 mammals) having benign SPNs. The reference level can be determined by imaging SPN tissues in the mammals after administering a labeled molecule having the ability to bind an αᵥβ₃ integrin polypeptide, and then taking biopsies of SPN tissue to determine whether they are benign or malignant. Any method can be used to determine whether the biopsy samples are benign or malignant. For example, the histology of the biopsy samples can be evaluated to determine whether the samples are benign or malignant. The levels of binding of a labeled molecule to the benign SPN tissues, as determined by imaging the benign SPN tissues in the mammals, can then be averaged to calculate the reference level of binding.

It will be appreciated that levels from comparable samples measured using comparable techniques can be used when determining whether or not a particular level is an elevated level. For example, the level of binding of a labeled molecule to a human sample can be compared to a reference level for human samples. In addition, the level of binding of a particular molecule call be compared to a reference level determined using that molecule, and the level of binding assessed using a particular technique (e.g., PET scanning) can be compared to a reference level determined using the same technique.

A labeled molecule having the ability to bind an αᵥβ₃ integrin polypeptide also can be used to distinguish benign SPN tissue from malignant SPN tissue following removal of SPN tissue from a mammal. For example, SPN tissue can be removed from a mammal (e.g., a human) having a SPN using a bronchoscope. In some cases, SPN tissue can be surgically removed from a mammal. Following removal of the SPN tissue from a mammal, the tissue can be immediately analyzed or preserved for future analysis. For example, the SPN tissue can be flash frozen in liquid nitrogen and stored at −80° C. In some cases, the tissue can be fixed in formalin, embedded in paraffin, and stored at room temperature for future analysis. Any appropriate method can be used to analyze SPN tissue with a labeled molecule that binds to cells having αᵥβ₃ integrin polypeptides. For example, SPN tissue can be sectioned, and analyzed by immunohistochemistry using an anti-αᵥβ₃ integrin antibody that binds to cells having αᵥβ₃ integrin polypeptides. The level of binding of the anti-αᵥβ₃ integrin antibody to SPN tissue can be used to determine whether the SPN tissue is malignant or benign. For example, the presence of the anti-αᵥβ₃ integrin antibody bound to SPN tissue can indicate that the SPN tissue is malignant, and the absence of the anti-αᵥβ₃ integrin antibody bound to SPN tissue can indicate that the SPN tissue is benign. In some cases, a detectable level of a labeled molecule that binds to an αᵥβ₃ integrin polypeptide (e.g., a labeled anti-αᵥβ₃ integrin antibody) bound to SPN tissue can indicate that the SPN tissue is malignant, and an undetectable level of a labeled molecule that binds to an αᵥβ₃ integrin polypeptide (e.g., a labeled anti-αᵥβ₃ integrin antibody) bound to SPN tissue can indicate that the SPN tissue is benign. In some cases, the presence of an elevated level of expression of αᵥβ₃ integrin polypeptides by SPN tissue as determined by, for example, assessing the binding of an anti-αᵥβ₃ integrin antibody to SPN tissue can indicate that the SPN tissue is malignant. In some cases, the absence of an elevated level of expression of αᵥβ₃ integrin polypeptides by SPN tissue as determined by, for example, assessing the binding of an anti-αᵥβ₃ integrin antibody to SPN tissue can indicate that the SPN tissue is benign.

Any appropriate method can be used to determine the level of expression of an αᵥβ₃ integrin polypeptide present in SPN cells or tissues. For example, the level of expression of an αᵥβ₃ integrin polypeptide can be determined using polypeptide detection methods such as Western blotting and immunochemistry techniques. The level of expression of an αᵥβ₃ integrin polypeptide present in SPN cells or tissues also can be determined by measuring the level of an RNA that encodes an αᵥβ₃ integrin polypeptide. Any appropriate method can be used to measure the level of an RNA that encodes an αᵥβ₃ integrin polypeptide including, without limitation, in situ hybridization and PCR-based methods.

This document also provides methods and materials to assist medical or research professionals in determining whether SPN tissue is malignant or benign. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of an αᵥβ₃ integrin polypeptide in a sample, and (2) communicating information about that level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

An SPN in a mammal can be monitored over time. For example, SPNs initially found to be benign can be monitored over time to determine whether or not they become malignant. In some cases, malignant SPNs that are not surgically removed can be monitored over time to determine whether or not they become larger. SPNs can be monitored regularly, such as every month, every three months, every six months, or every twelve months. SPNs can also be monitored on an irregular basis. In addition, SPNs can be monitored for any duration of time, such as months or years. In some cases, SPNs can be monitored for the duration of a lifetime.

Methods and materials provided herein can be used in combination with standard methods of diagnosing lung cancer to assess SPNs in a mammal. For example, methods and materials provided herein can be used in combination with findings from a patient history, physical examination, chest x-ray, PET scan, and/or CT scan to assess SPNs in a mammal. In some cases, methods and materials provided herein can be used in combination with histological evaluations (e.g., performed by a pathologist) to assess SPNs in a mammal.

The invention will be further described in the following examples, which do not limit the scope of the invention described in the claims.

### EXAMPLES

### Example 1—Immunohistochemical Analysis of Lung Tissue for αᵥβ₃ Integrin Expression

Lung cancer (non-small cell bronchoalveolar cell and small cell) and granuloma lung tissue were obtained from the Mayo Lung Cancer Tissue Registry. Paraffin embedded specimens were used to perform immunohistochemistry, probing for αᵥβ₃ integrin expression. After routine processing of lung sections, which included preheated 1 mM EDTA, pH 8.0 retrieval for 30 minutes, the sections were incubated in 1:100 αᵥβ₃ antibody (LM609, Chemicon International) for 30 minutes. Detection was completed by the use of a biotin-free polymer, Rabbit MACH3 (Biocare Medicals, Walnut Creek, Calif.) for 10 minutes. Nova Red (Vector Laboratories) and Modified Schmidts's Hematoxylin counterstain was used as the chromogen, and sections were mounted with a permanent mounting media.

A high level of αᵥβ₃ integrin expression was observed on the surface of lung cancer cells, whereas little to no expression of αᵥβ₃ integrin was observed on benign granuloma cells.

### Example 2—Detecting Malignant Pulmonary Nodules in a Clinical Study

The following clinical study is performed to verify the improved sensitivity and specificity of using αᵥβ₃ integrin expression to differentiate benign SPNs from malignant SPNs. The primary endpoint is diagnostic accuracy. Patients are recruited from an active patient population. Management algorithms are typically based on clinical experience, radiologic appearance, rate of change in nodule size with observation, patient preference, and the need to expedite surgery for malignancy and avoid intervention for benign nodules. There are a number of other tests used to help guide management including transthoracic lung biopsy, CT contrast enhancement, and PET scan.

Subjects are patients presenting for evaluation of indeterminate pulmonary nodules. After consent, they are randomized to either of two study groups; one, with no intervention (i.e., physicians' usual care which might include observation or other diagnostics testing as noted herein); the second, usual clinical practice but including testing αᵥβ₃ integrin expression. The diagnostic accuracy of the αᵥβ₃ integrin expression testing is determined by following these groups until diagnosis of the nodule is made.

Image analysis is performed by one of three experienced radiologists. They are blinded to the subject's clinical history and other test results. Diagnostic accuracy is assessed in comparison to the final diagnosis of the SPN treated by usual care. Sensitivity, specificity, positive, and negative predictive values are calculated.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a solitary pulmonary nodule, said method comprising determining whether or not said solitary pulmonary nodule expresses an αᵥβ₃ integrin polypeptide, wherein the expression of said αᵥβ₃ integrin polypeptide indicates that said solitary pulmonary nodule is malignant, and wherein the expression of little or no αᵥβ₃ integrin polypeptide indicates that said solitary pulmonary nodule is benign.

2. The method of claim 1, wherein said solitary pulmonary nodule is a human solitary pulmonary nodule.

3. The method of claim 1, wherein said determining step comprises contacting tissue of said solitary pulmonary nodule with a labeled molecule having the ability to bind to said αᵥβ₃ integrin polypeptide.

4. The method of claim 3, wherein said labeled molecule is an antibody.

5. The method of claim 3, wherein said labeled molecule is an RGD peptide.

6. The method of claim 3, wherein said labeled molecule is ^{99m}Tc-NC100692.

7. The method of claim 3, wherein said labeled molecule is administered to a mammal having said solitary pulmonary nodule.

8. A method for assessing a solitary pulmonary nodule, said method comprising determining whether or not said solitary pulmonary nodule lacks expression of an αᵥβ₃ integrin polypeptide, wherein the lack of expression of said αᵥβ₃ integrin polypeptide indicates that said solitary pulmonary nodule is benign.

9. The method of claim 8, wherein said solitary pulmonary nodule is a human solitary pulmonary nodule.

10. The method of claim 8, wherein said determining step comprises contacting tissue of said solitary pulmonary nodule with a labeled molecule having the ability to bind to said αᵥβ₃ integrin polypeptide.

11. The method of claim 10, wherein said labeled molecule is an antibody.

12. The method of claim I 0, wherein said labeled molecule is an RGD peptide.

13. The method of claim 10, wherein said labeled molecule is ^{99m}Tc-NC100692.

14. The method of claim 10, wherein said labeled molecule is administered to a mammal having said solitary pulmonary nodule.
